(19) United States
(12) Patent Application Publication
(10) Pub. No.: US 2010/0086625 A1
(43) Pub. Date: Apr. 8, 2010

(54) METHODS FOR TREATING SKIN LESIONS

(76) Inventor: Abdul R. Abukurah, Northridge, CA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 12/575,232

(22) Filed: Oct. 7, 2009

Related U.S. Application Data
(60) Provisional application No. 61/103,854, filed on Oct. 8, 2008.

(51) Int. Cl. A61K 33/06

(52) U.S. Cl. 424/682

(57) ABSTRACT

The present invention relates to the treatment of skin lesions such as cold sores and other complications resulting from disorders such as herpes and the like. The invention relates to the use of a composition comprising one or more aluminum salts for treatment purposes.

### CROSS-REFERENCE TO RELATED APPLICATION

This application claims the benefit U.S. Provisional Application No. 61/103,854, filed Oct. 8, 2008, which is incorporated herein by reference in its entirety.

### BACKGROUND

Most skin lesions are caused by viral infections such as an infection by herpes viruses. There are two types of herpes simplex virus: herpes simplex virus 1 (HSV-1) and herpes simplex virus 2 (HSV-2). Cold sores or fever blisters are often caused by simplex virus 1. Genital herpes are often caused by simplex virus 2.

Cold sores or fever blisters are characterized by a blister or groups of blisters containing a clear fluid formed on the skin or mucous membranes such as the lips or mouth. In addition, cold sores or fever blisters may be accompanied by a cold or fever or both. The blister associated with this disease may cover a large portion of the lip and cause severe itching, stinging, and localized pain. Without medication, it is common for a blister to remain on the skin in excess of one week and to take two weeks to completely heal. In some instances where secondary infections occur, the healing period can be even further prolonged.

There are several medications available to treat cold sores. Some are used topically and others are taken orally. For example, penciclovir 1% cream (Denavir®) is believed to reduce the time to healing by two days if starting treatment within one hour of an outbreak; frequent application of acyclovir 5% cream (Zovirax®) is believed to reduce the time to healing by about half a day; taking famciclovir (Famvir®) 1500 mg may shorten the herpes infection by two days; and taking valacyclovir (Valtrex®) 2 gm twice a day for one day may shorten a herpes infection by a little over one day.

Sometimes, local anesthetics are used to mitigate pain associated with such sores, and antibiotics are used to control secondary bacterial infections when they occur. Ointments may also be used to soften crusts. However, use of an antibiotic frequently causes side reactions and tends to sensitize the patient to further effective or safe use of the drug. Hence, it is important to avoid the secondary infection stage. Until the present invention, it is not believed that an effective cure has been available.

Therefore, there is a need for an effective method of treating skin lesions caused by viral infection, such as fever blisters or cold sores. The present invention seeks to address this need and provide further related advantages.

### SUMMARY OF THE INVENTION

The present invention relates to a novel method for treating skin lesions. For example, provided is a method of treating a skin lesion in a subject in need of treatment, comprising topically administering to said lesion a composition comprising an effective amount of one or more aluminum salts. Also provided is a method of treating a skin lesion in a subject in need of treatment, comprising topically administering to said lesion a composition consisting essentially of an effective amount of one or more aluminum salts. Embodiments also provide a method of treating a skin lesion in a subject in need of treatment, comprising topically administering to said lesion a composition consisting of an effective amount of one or more aluminum salts.

The aluminum salt may be selected from a group consisting of potassium aluminum sulfate, anhydrous potassium aluminum sulfate, potassium aluminum sulfate dodecahydrate, ammonium aluminum sulfate, anhydrous ammonium aluminum sulfate, and ammonium aluminum sulfate dodecahydrate.

A composition of the present invention may further comprise an analgesic, such as lidocaine or benzocaine. A composition may further comprise a pharmaceutically acceptable carrier. The composition may be comprised in a gel, cream, lotion, or liquid formulation. A composition of the present invention may be held in a collapsible container, for example.

A composition may be applied directly to a skin lesion, and the topical application may be repeated on the subject in need of the treatment twice a day for two to five days.

Also provided is a method comprising the steps of wetting a styptic pencil with water to provide a wetted styptic pencil and applying the wetted styptic pencil directly on a skin lesion. Also provided is a method that consists essentially of the steps of wetting a styptic pencil with water to provide a wetted styptic pencil; and applying the wetted styptic pencil directly on a skin lesion. Further provided is a method that consists of the steps of wetting a styptic pencil with water to provide a wetted styptic pencil; and applying the wetted styptic pencil directly on a skin lesion.

Also contemplated is a method comprising the steps of wetting styptic powder with water to provide a styptic powder solution; and applying the styptic powder solution directly on a skin lesion. Provided further is a method consisting essentially of the steps of wetting styptic powder with water to provide a styptic powder solution; and applying the styptic powder solution directly on a skin lesion. Also provided is a method consisting of the steps of wetting styptic powder with water to provide a styptic powder solution; and applying the styptic powder solution directly on a skin lesion.

Another aspect contemplates a method of treating a skin lesion in a subject in need of treatment, comprising spraying a solution comprising an about 50% (w/v) concentration of one or more aluminum salts on the lesion. Also provided is a method of treating a skin lesion in a subject in need of treatment, comprising spraying a solution consisting essentially of an about 50% (w/v) concentration of one or more aluminum salts on the lesion. Also provided is a method of treating a skin lesion in a subject in need of treatment, comprising spraying a solution consisting of an about 50% (w/v) concentration of one or more aluminum salts on the lesion.

Methods of the present invention are useful in treating skin lesions, including viral-caused skin lesions such as a herpes lesion or a cold sore.

### DETAILED DESCRIPTION OF THE INVENTION

The present invention is related to a novel method for treating skin lesions. In one aspect, the present invention provides a method of treating a skin lesion, such as lesions caused by a herpes virus. Persons of skill in the art are familiar with methods of identifying skin lesions of the type discussed herein.

A method may comprise the step of topically administering to a skin lesion a pharmacologically effective amount of a composition comprising one or more aluminum salts. A method may comprise treating a skin lesion in a subject in need of treatment, comprising topically administering to said lesion a composition comprising, consisting essentially of, or consisting of an effective amount of one or more aluminum salts.

A person skilled in the art will recognize that the aluminum salt useful in the present invention may be an aluminum salt in any form including, but not limited to, potassium aluminum sulfate and ammonium aluminum sulfate. Potassium aluminum sulfate may be in either anhydrous form or any hydrate form. The aluminum salt may be potassium aluminum sulfate dodecahydrate, KAI(SO₄)₂.12H₂O. Similarly, ammonium aluminum sulfate may be in either anhydrous form or any hydrate form. The aluminum salt may be ammonium aluminum sulfate dodecahydrate, NH₄Al(SO₄)₂.12H₂O. Aluminum salt suppliers are well-known in the art (e.g., Hospira, Inc., or Abbott Laboratories).

Compositions of the present invention may comprise an effective amount of one or more aluminum salts and may optionally include additional agents, as discussed below. As used herein, the term “effective” (e.g., “an effective amount”) means adequate to accomplish a desired, expected, or intended result. For example, an effective amount may refer to an amount necessary to reduce the appearance of a skin lesion. As used herein, a “pharmacologically effective amount” refers to that amount of the compound effective to produce the intended pharmacological result, e.g., reduce the appearance of a skin lesion, reduce pain, or reduce stinging. As such, an “effective amount” may be the same as a “pharmacologically effective amount.”

A composition of the present invention may comprise, consist essentially of, or consist of one or more aluminum salts in an amount of about 5-100% (w/v) or (w/w). In certain embodiments, the amount is about, at most about, or at least about 5, 10, 20, 30, 40, 50, 60, 70, 80, 90, 95, or 100%, or any range derivable therein. In certain embodiments, the amount is about 25-50%. In certain embodiments, the amount is about 40-60%. In particular embodiments, the amount is about 50%. For example, a solution may comprise a 50% (w/v) amount of an aluminum salt, such as ammonium aluminum sulfate dodecahydrate. As another example, a styptic pencil may comprise 100% of a salt described herein.

A composition may further comprise an analgesic to reduce the pain caused by the skin lesion. Analgesics useful in the present invention may include lidocaine or lidocaine HCl, benzocaine, butamben picrate, dibucaine or dibucaine HCl, dimethisoquin HCl, dyclonine HCl, pramoxine HCl, tetracaine or tetracaine HCl.

Antibiotic agents may be added to a composition to control a secondary bacterial infection resulting from the skin lesion. A person skilled in the art will recognize that many topical antibiotic agents may be useful in the present invention, including, but not limited to, polymixin B sulfate, gramicidin, bacitracin, and bacitracin zinc.

Other ingredients may be added to a composition for application ease such as increasing the softness and improving the smell of the formulation. Therefore, the composition may further comprise a pharmaceutically acceptable carrier. The phrases “pharmaceutically or pharmacologically acceptable” refers to molecular entities and compositions that do not produce an adverse, allergic, or other untoward reaction when administered to a subject.

The carrier component should be physiologically compatible with human skin and membrane tissues. The carrier may be a mixture of components. It is typically non-irritating and may act as a thickening agent for the composition. A number of such agents are well known in the art, for example, the CTFA Cosmetic Ingredient Dictionary. Carrier components may also act as a solvent for other components of the composition. In addition, the carrier may act as a demulcent and may have emollient properties. Some typical carrier components include glycerin and pectin. Glycerin is a viscous hygroscopic liquid that acts as a solvent and demulcent. Glycerin may be present at a concentration of 15-40% v/v, for example, although concentrations of glycerin may be varied over an even broader range to achieve a desired consistency.

Another optional carrier component may be pectin. Pectin is a negatively charged polysaccharide that adds viscosity to a composition. Typically, pectin is included at a concentration of 0.5%-2% w/v; however, as for glycerin above, the concentration may be considerably varied to achieve a desired consistency. Other typical carrier components include gums, mucilages, dextrins, and hydroxy- and carboxycelluloses. Carrier components may be added that provide (i) an agreeable feeling (such as camphor or phenol), (ii) improved fluidity (such as polyalcohols), or (iii) improved storage stability (such as sodium benzoate). Except insofar as any conventional carrier is incompatible with the active ingredient, its use in the therapeutic or pharmaceutical compositions discussed herein is contemplated.

A number of preservatives are available for use in the composition described herein, including: benzalkonium chloride, organic mercury compounds, sorbic acid, hexachlorophene, and parabens. One exemplary preservative is sodium benzoate. Sodium benzoate may be used in compositions of the present invention at a concentration of about 0.1-0.50%, such as 0.25% w/v, for example.

Methods of formulation are well known in the art and are disclosed, for example, in Remington: The Science and Practice of Pharmacy, Mack Publishing Company, Easton, Pa., 19th Edition (1995). Pharmaceutical formulations for topical administration of a composition include ointments, pastes, creams, lotions, gels, powders, solutions, sprays, inhalants, or patches. An active component (e.g., an aluminum salt) is typically admixed under sterile conditions with a pharmaceutically acceptable carrier and any needed preservatives or buffers as may be required. A composition may be administered by rubbing a cream comprising the composition on the lesion, for example, or by spraying an aqueous solution comprising the composition on the lesion. Other administration methods are discussed herein. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

A product including the composition of the present invention may be held in a collapsible container as a liquid, cream, lotion, ointment, or gel form. Alternatively, the product may be in a form of stick. The package of the product may bear the warning such as “do not use this product on or around the eye in any form or shape.” A liquid composition may be comprised in a spray bottle, such as a 30 cc spray bottle.

The product may be applied directly on the cold sore or herpes lesion on the lips or inside the mouth, or on the external or internal vaginal canal, or on the penile shaft, or on any herpes sore on the outside skin. The topical application of the product may be repeated on the subject in need of the treatment, such as for twice a day for two to five days. Administration may take place two times a day for 2, 3, 4, or 5 days, or any range derivable therein, for example. The sore will likely sting at the time of the application, which will typically last for about 1-2 minutes. The sting will typically go away after the second day of application.

Another aspect of the present invention contemplates a method of treating a skin lesion in a subject in need of treatment, comprising spraying a solution consisting essentially of an about 50% (w/v) concentration of one or more aluminum salts on the lesion.

Styptic or hemostatic pencils and styptic powder are known to include an aluminum salt. A styptic or hemostatic pencil is a short stick of medication, usually anhydrous aluminum sulfate or titanium dioxide, which is used for staunching blood by causing blood vessels to contract at the site of the wound. Styptic powder is usually used to stop bleeding from nails that are clipped too closely.

The inventors of the present invention unexpectedly discovered that a styptic pencil or styptic powder is effective in treating skin lesions, such as cold sores. In one embodiment, the present invention therefore provides a method for treating a skin lesion comprising the steps of wetting a styptic pencil with water to provide a wetted styptic pencil and applying the wetted styptic pencil directly on a skin lesion. In another embodiment, the present invention provides a method for treating a skin lesion, including the step of wetting styptic powder with water to provide a styptic powder solution and applying the styptic powder solution directly on a skin lesion. Any pencil or powder discussed herein comprises an effective amount of the salt(s) for the purpose of treating a skin lesion.

The method of using the styptic pencil is to wet the styptic stick with water and apply it directly on the cold sore or herpes lesion. The application may be used twice a day for 2, 3, 4, or 5 days, or any range derivable therein. Styptic powder may also be applied with this frequency.

As used herein, the term “patient” or “subject” refers to a living mammalian organism, such as a human.

“Treatment” and “treating” as used herein refer to administration or application of an active ingredient, such as one or more aluminum salts, to a patient or performance of a procedure or modality on a patient for the purpose of obtaining a therapeutic benefit of a disease or health-related condition. For example, a patient having a skin lesion may be subjected to a treatment comprising administration of a composition comprising one or more aluminum salts in order to reduce the appearance of the skin lesion or to minimize conditions associated with the lesion, such as pain.

The term “therapeutic benefit” as used throughout this application refers to anything that promotes or enhances the well-being of the patient with respect to the medical treatment of a condition. This includes, but is not limited to, a reduction in the onset, frequency, duration, or severity of the signs or symptoms of a viral-caused skin lesion.

In any embodiment herein, the term “comprising” may be substituted with “consisting essentially of” or “consisting of.” For example, the present invention contemplates a method of treating a skin lesion in a subject in need of treatment consisting essentially of topically administering to said lesion a composition comprising an effective amount of one or more aluminum salts. As another example, the present invention contemplates a method of treating a skin lesion in a subject in need of treatment consisting essentially of topically administering to said lesion a composition consisting essentially of an effective amount of one or more aluminum salts. The present invention also contemplates a method of treating a skin lesion in a subject in need of treatment consisting of topically administering to said lesion a composition consisting essentially of an effective amount of one or more aluminum salts. The present invention further contemplates a method of treating a skin lesion in a subject in need of treatment consisting of topically administering to said lesion a composition consisting of an effective amount of one or more aluminum salts. Other similar substitutions in any other embodiment discussed herein are also encompassed by the present invention.

For those embodiments reciting “consisting essentially of,” it is noted that non-limiting examples of materials and steps that do not materially affect the basic and novel aspects of an aluminum salt(s) include those that do not change the chemical structure of the aluminum salt(s) employed, that do not interfere with access of the aluminum salt(s) to the lesion, or those that do not decrease the effective amount of the aluminum salt(s) that is administered. Any further ingredient described herein (e.g., a carrier, a preservative, an antibiotic, an analgesic, an excipient) may be combined in a composition that comprises, consists essentially of, or consists of one or more aluminum salts.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

As used herein, “a” or “an” means one or more, unless clearly indicated otherwise.

Throughout this application, the term “about” is used to indicate that a value includes the standard deviation of error for the device and/or method being employed to determine the value.

### EXAMPLE 1

An aqueous solution of 50% (w/v) ammonium aluminum sulfate dodecahydrate was applied to a lesion in the mouth of a patient (a cold sore) the day following discovery of the lesion. The solution was applied as a spray twice a day every 12 hours for three days. Upon application, a stinging sensation was experienced by the patient for about one minute. Future applications did not produce a stinging sensation. By the third day, the lesion had regressed and resolved itself. This patient's cold sores normally lasted seven days or more before healing without treatment.

### EXAMPLE 2

An aqueous solution of 50% (w/v) ammonium aluminum sulfate dodecahydrate was applied to a fever blister experienced by a patient. Sores became smaller after the first application (by spraying of the solution) and application of the solution twice a day for two days caused the blister to disappear.

### EXAMPLE 3

An aqueous solution of 50% (w/v) ammonium aluminum sulfate dodecahydrate was applied to a herpes simplex outbreak experienced by a patient. Starting with the first application of the solution by spraying, the outbreak began to dry up. Following twice-a-day applications, the outbreak had disappeared by the fourth day. This patient's outbreak typically takes 1-2 weeks to disappear without treatment.

While illustrative embodiments are illustrated and described herein, it will be appreciated that various changes can be made therein without departing from the spirit and scope of the invention.

1. A method of treating a skin lesion in a subject in need of treatment, comprising topically administering to said lesion a composition consisting essentially of an effective amount of one or more aluminum salts.

2. The method of claim 1, wherein the aluminum salt is selected from a group consisting of potassium aluminum sulfate, potassium aluminum sulfate dodecahydrate, ammonium aluminum sulfate, and ammonium aluminum sulfate dodecahydrate.

3. The method of claim 2, wherein the concentration of the aluminum salt in the composition is about 40-60%.

4. The method of claim 3, wherein the concentration of the aluminum salt in the composition is about 50%.

5. The method of claim 1, wherein the composition further comprises an analgesic.

6. The method of claim 5, wherein the analgesic is selected from the group consisting of lidocaine, benzocaine, butamben picrate, dibucaine, dimethisoquin HCl, dyclonine HCl, pramoxine HCl, and tetracaine.

7. The method of claim 1, wherein the composition further comprises a pharmaceutically acceptable carrier.

8. The method of claim 1, wherein the composition is in a gel formulation.

9. The method of claim 1, wherein the composition is in a cream formulation.

10. The method of claim 1, wherein the composition is in a lotion formulation.

11. The method of claim 1, wherein the composition is in a liquid formulation.

12. The method of claim 11, wherein the composition is topically administered by spraying.

13. The method of claim 1, wherein the composition is held in a collapsible container.

14. The method of claim 1, wherein the topical application is repeated on the subject in need of the treatment twice a day for two to five days.

15. A method of treating a skin lesion in a subject in need of treatment, consisting essentially of:
wetting a styptic pencil with water to provide a wetted styptic pencil; and
applying the wetted styptic pencil directly on a skin lesion.

16. The method of claim 15, wherein the skin lesion is a viral-caused skin lesion.

17. The method of claim 15, wherein the skin lesion is a herpes lesion.

18. The method of claim 15, wherein the skin lesion is a cold sore.

19. A method of treating a skin lesion in a subject in need of treatment, consisting essentially of:
wetting styptic powder with water to provide a styptic powder solution; and
applying the styptic powder solution directly on a skin lesion.

20. The method of claim 19, wherein the skin lesion is a viral-caused skin lesion.

21. The method of claim 19, wherein the skin lesion is a herpes lesion.

22. The method of claim 19, wherein the skin lesion is a cold sore.

23. A method of treating a skin lesion in a subject in need of treatment, comprising spraying a solution consisting essentially of about 50% (w/v) concentration of one or more aluminum salts on the lesion.
